(19) United States
(12) Patent Application Publication
(10) Pub. No.: US 2017/0231565 A1
(43) Pub. Date: Aug. 17, 2017

(54) Vital Sleeve

(71) Applicant: Antionette Olivarez, San Fernando, CA (US)

(72) Inventor: Antionette Olivarez, San Fernando, CA (US)

(21) Appl. No.: 15/044,098

(22) Filed: Feb. 15, 2016

(51) Int. Cl. A61B 5/00; A61B 5/0205; A61M 27/00; A41D 13/08; A61F 15/00

(52) U.S. Cl. CPC A61B 5/6802; A41D 13/08; A61F 15/004; A61M 27/002; A61B 5/02055; A61B 5/742; A61B 5/486; A61B 5/0022; A61B 5/02438

(57) ABSTRACT

The present invention discloses a medical sleeve arm to protect the arm having fistula or shunt from any external assault. The sleeve is configured to be worn in arm and included an inner cushioning layer and outer durable layer to provide protection against bumps and physical damage. The medical sleeve arm further comprises means to monitor vital signs of the person that may help to detect any potential problem in the fistula or shunt. Furthermore, the vital signs monitored could be remotely communicated to a remote computing device that allows a concerned medical personal to observe the vital signs normally and during the dialysis.

### CROSS REFERENCE TO RELATED APPLICATION

Not Applicable

### FEDERALLY SPONSORED RESEARCH AND DEVELOPMENT

Not Applicable

### MICROFICHE APPENDIX

Not Applicable

### BACKGROUND OF THE INVENTION

(1) Field of the Invention

The present invention generally relates to medical arm sleeve and in particular an arm sleeve for monitoring vital signs and protecting the shunt or fistula site in the arm of a patient.

(2) Background of the Invention

Patients who have an injury to a particular area, or site, on the body which requires a relatively long healing time and/or protection from external elements, or who have a medical condition which requires access to a port site for periodic administration of medication, often have difficulty protecting these sites from contamination or damage due to external influences. These sites are usually fragile and painful to touch or pressure. One such site may be a wound, perhaps requiring stitches or administration of topical medication under a sterile dressing to facilitate the healing process. However, exposure to external elements is often detrimental to the healing process, even when the site is covered by a sterile dressing.

Likewise, before starting hemodialysis treatments, one important step is preparing a vascular access, which is the site on your body where blood will be removed and returned during dialysis. For most people, an arteriovenous (AV) fistula is the best kind of vascular access. An AV fistula is created surgically by connecting an artery directly to a vein, usually in the forearm of patients. Then the vein grows larger and stronger, making repeated needle insertions easier.

However, the regular hemodialysis sessions occur 2-3 times per week. In the long run, there could be hundreds of pinholes concentrate on the fistula which is only several centimeters in length. Thus, the repeated use of the fistula is likely to cause clots or infection and that may result in frequent replacement of fistula, via surgical intervention. The general way for improving the life of fistula/shunt and protecting the arm with fistula thereof is to use the arm for hemodialysis and not for other medical procedures. Further, avoiding any external pressure or assaults on arm may help to keep the arm healthy and increase the life of fistula/stunt.

Accordingly, various prior arts have disclosed different type of protective sleeves to protect such sites. For example, U.S. Pat. No. 8,419,638 discloses implantable devices for evaluating body-associated fluid transport structures and method of using the same, wherein the implantable devices include an elongated structure with at least one hermetically sealed integrated circuit sensor stably associated therewith and a transmitter. Another, U.S. Pat. No. 6,276,364 discloses a protective sleeve for a chronically implanted intravenous site (IV) site, wherein the sleeve is made of a prophylactic waterproof material, and is normally worn by a person while showering to prevent moisture from invading the IV site. Yet another U.S. Pat. No. 5,592,953 discloses protective sleeves for protectively covering an appendage and medical sites such as intravenous equipment, bandages, wounds, etc. The sleeve is open at both ends with adjustable elastic sections, which effectively seal the sleeve against the appendage. Still another U.S. Pat. No. 6,267,115 discloses a protective device used for an intravenous (IV) site during intravenous infusion includes a waterproof flexible wrap that wraps around a portion of a limb where an intravenous catheter enters a vein. Another Patent No US20030139792discloses a fistula thermotherapy device, especially to a fistula thermotherapy device that not only protects the arm with the fistula for hemodialysis, but also facilitates the metabolism of the cells near the needle insertions on fistula.

Therefore, to avoid unnecessary suffering of patients due to shunt/fistula placement and reduce the workload of health care personnel, providing a sleeve to protect the arm from external assaults and further providing monitoring of vital signs could help to keep the arm of patient health and avoid any further complication or discomfort to the patient due to fistula/shunt.

### SUMMARY OF INVENTION

In view of the foregoing disadvantages inherent in the known types of protective sleeves or like in the prior art, the present invention provides an improved vital sleeve that protects the shunt site as well as monitor certain parameters of the body as well. As such, the general purpose of the present invention, which will be described subsequently in greater detail, is to provide a new and improved medical arm sleeve with all the advantages of the prior art and none of the disadvantages.

Accordingly, it is an object of the present invention to provide a medical arm sleeve to protect the fistula/shunt site in arm from external assaults and prevent any kind of infection or other damages caused to the arm.

Another object of the present invention is that the medical arm sleeve that monitors vital signs for keeping the arm healthy and early detection of any abnormal functioning.

A further object of the present invention is that the medical arm sleeve allows remote monitoring of the vital signs.

A still further object of present invention is that the medical arm sleeve is light in weight.

An additional object of present invention is that the medical arm sleeve is comfortable to skin.

Yet another object of present invention is that the medical arm sleeve is easy to wear.

There has thus been outlined, rather broadly, the more important features of the invention in order that the detailed description thereof that follows may be better understood and in order that the present contribution to the art may be better appreciated.

Numerous objects, features, and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. In addition, it is to be understood that the phraseology and terminology employed herein are for the purpose of descriptions and should not be regarded as limiting.

### BRIEF DESCRIPTION OF THE DRAWINGS

To further clarify various aspects of some example embodiments of the present invention, a more particular description of the invention will be rendered by reference to specific embodiments thereof, which are illustrated in the appended drawing. It is appreciated that the drawing depicts only illustrated embodiments of the invention and are therefore not to be considered limiting of its scope. The invention will be described and explained with additional specificity and detail through the use of the accompanying drawing in which:

FIG. 1 is the perspective view of medical arm sleeve worn on arm.

FIG. 2 is the perspective view of medical arm sleeve in the open position.

FIG. 3 is the bottom view of the medical sleeve arm showing the showing the display screen and control buttons.

### DETAILED DESCRIPTION OF THE INVENTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention, and together with the description, serve to explain the principles of the invention. To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

In some preferred embodiments, the present invention provides a medical arm sleeve adapted to be worn in arm of a person having implanted fistula/shunt in the arm. The purpose of the invention is to provide a protective, user friendly, easy to wear sleeve for the arm.

This section summarizes some aspects of the present disclosure and briefly introduces some preferred embodiments. Simplifications or omissions in this section as well as in the abstract or the title of this description may be made to avoid obscuring the purpose of this section, the abstract, and the title. Such simplifications or omissions are not intended to limit the scope of the present disclosure nor imply any limitations.

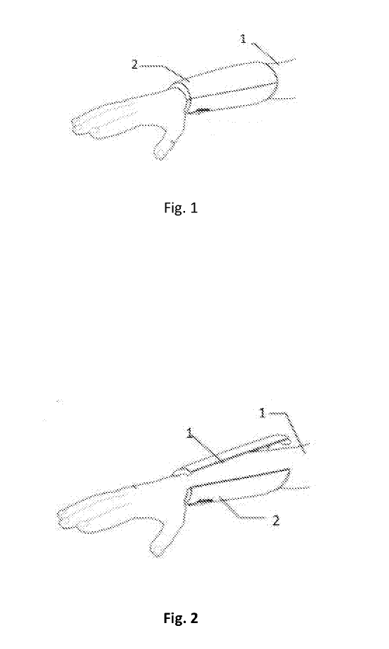

Turning now descriptively to drawing and referring to FIG. 1-3, which shows a medical arm sleeve worn in the arm 1. The sleeve 2 aims to protect the arm portion having an implanted fistula or shunt against the external assaults. The rectangular shaped sleeve is configured to be worn in a human arm by placing the sleeve over the arm as shown in FIG. 2. The sleeve has fasteners on opposite sides for securing the sleeve over the arm. The fasteners could be hook and loop fastener, snap fit or plug type fasteners wherein one edge of the sleeve includes a male part of the fastener and the opposite edge of sleeve contains a female part of fastener. To secure the sleeve to the arm, the arm is places inside the sleeve with the two open edges of the sleeve facing each other, and one of the edge is pushed towards the other to secure the fastener.

The inner layer of sleeve is made of skin friendly material that is comfortable to skin and may further has moisture wicking properties. Further cushioning is provided in the inner layer for additional comfort and a level of protection against external assaults such as bumps and physical damage on the arm. The outer layer of sleeve is durable to provide additional protection to the arm. Thus, the sleeve fully covers the fistula/shunt site and the person does not have to worry about keeping the arm safe and protected.

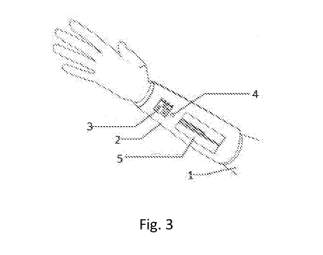

In a preferred embodiment, the disclosed medical arm sleeve further include additional feature for monitoring vital signs at the fistula/shunt site. The vital signs include blood pressure, body temperature, pulse rate of the shunt etc. The monitoring of the vital signs helps for early detection of any potential problems to the fistula/shunt. As shown in FIG. 3 the sleeve is provided with a display screen 3 and an inner measuring analytes incorporated within the sleeve, provided the same will monitor pulse or thrill and blood flow rate and display the measured values on the display panel. The display panel could help the patient as well as the doctor to monitor and analyze the parameters and treat the site in case of emergency. An on/off switch 4 is provided to turn the monitoring of vital signs on and off. The button is a simple push type button that could be pressed to start the monitoring and it could be pressed again to turn the monitoring off. The sleeve is further provided with a window 5 positioned over the fistula/shunt site and provide access to the fistula or shunt during dialysis. Preferably, the window has a transparent cover that allows viewing the fistula/shunt site.

In another preferred embodiment, the disclosed sleeve with additional features consisting of connecting the sleeve with a remote computing device to allow the physician to use tele monitoring, and remain updated about the health parameters of the person. The ability of the sleeve to connect to the visual monitor allows for convenient tele monitoring of the shunt site, even if the physician is unable to reach the patient site. The ability to communicate the health parameters to a remote device in real-time allows a concerned medical personal to observer the dialysis remotely, such as from his office and take appropriate action in case any problem occur during dialysis. Thus, the sleeve is capable enough to serve dual functions: protecting the shunt site from any external damage and monitoring of the physiological parameters at the shunt site with convenience to the patient as well as physician.

Additionally, the sleeve is designed to fit in the arm of a person, wherein the physician or supplier could obtain the unique measurements of the person and the exact location of the shunt on the arm. It is advisable to cast the sleeve accordingly, provided the sleeve does not spin about the arm, not constrict the arm at any point, but be able to monitor the vital signs of the patient. Additionally, the provided window should be wide enough for visualizing the shunt site.

In another embodiment, the medical arm sleeve of present invention protects the arm (shunt/fistula) from external assaults to reduce the risk of damage, thereby reducing the need for intervention/hospital readmission/missed dialysis/thrombus (clot)/surgery. This in turn may provide cost savings and increase quality for payers/providers/patients. Therefore, align the invention with the affordable care act/federal and state regulators and HHS, for improving quality outcomes and managing hemodialysis patients.

It is to be understood that the medical arm sleeve encompasses various alternative for example the sleeve could be provided in different shapes from square to rectangular. The vital signs monitoring could be varied for example, the sleeve could only provide protection to the arm with no vital sign monitoring; or the sleeve is could include technology that will measure the pulse and blood flow in the fistula. Further the sleeve could have advanced vital sign monitoring obvious to a skilled person. The sleeve could be equipped with a GPS device for emergency locating of hemodialysis patients as well. Furthermore, the medical sleeve arm may include various forms of indicia and color.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

I/We claim:

1. A protective sleeve for shunt site comprising:
(a) a basic sleeve;
(b) a covering site for shunt or fistula; and
(c) a display panel

2. The sleeve of claim 1, wherein the protective sleeve is to be worn in arm of the patient having implanted shut in order to protect the shunt site from external damage.

3. The sleeve of claim 1, wherein the protective sleeve will prevent any further contact with debris and environmental conditions.

4. The sleeve of claim 1, wherein the sleeve is designed in a manner so as to include additional feature to monitor various body parameters at the shunt site with the help of the same sleeve.

5. The sleeve of claim 1, wherein the display panel is provided on the upper side of the sleeve and measuring analytes on the inner side of the sleeve to monitor pulse rate, blood pressure and display the measured values on the display panel.

6. The sleeve of claim 1, wherein the display panel will help the patient as well as the physician to monitor and analyze the parameters and allow early intervention and treatment of the site in case of emergency.

7. The sleeve of claim 1, wherein the sleeve is further modified in a way to connect the sleeve to a computing device that allows the physician to use tele-monitoring and be updated about the status of the site without the need for the patient to leave his or her home.

8. The sleeve of claim 1, wherein the size of sleeve is customized for each individual.

9. The sleeve of claim 1, wherein the sleeve does not spin about the arm, not constrict the arm at any point.

10. The sleeve of claim 1, wherein the sleeve protects the arm containing shunt site from injury to reduce the risk of injury, thereby reducing the need for intervention/hospital readmission/missed dialysis/thrombus clot/surgery.
